(19) United States
(12) Patent Application Publication
(10) Pub. No.: US 2018/0221411 A1
(43) Pub. Date: Aug. 9, 2018

(54) Improved Cell Therapies

(71) Applicant: Cell Ideas Pty Ltd

(72) Inventors: Graham Vesey, Hornsby, New South Wales (AU); Elizabeth Evans, Greenwich, New South Whales (AU)

(21) Appl. No.: 15/537,427

(22) PCT Filed: Dec. 18, 2015
(86) PCT No.: PCT/AU2015/000755

§ 371 Date: Jun. 18, 2017

(30) Foreign Application Priority Data
Dec. 19, 2014 (AU) 2014905156
Dec. 19, 2014 (AU) 2014905157
May 26, 2015 (AU) 2015901929

(51) Int. Cl. A61K 35/28; A61P 29/00; A61P 19/02

(52) U.S. Cl. CPC A61K 35/28; A61P 19/02; A61P 29/00

(57) ABSTRACT

The present invention relates to methods for the improved treatment of an inflammatory disorder, where the treatment comprises administration of sex-matched stem or progenitor cells, such as mesenchymal stem cells (MSCs), or sex-matched cell secretions, or a combination thereof to a subject. The invention also relates to kits and compositions which may be used in such methods.

### PRIORITY CLAIM

This application claims priority to Australian provisional patent application number 2014905156 filed on 19 Dec. 2014, and to Australian provisional patent application number 2014905157 filed 19 Dec. 2014, and to Australian provisional patent application number 2015901929 filed 26 May 2015, the contents of each of which are hereby incorporated by reference.

### FIELD

The present invention relates to methods for the improved treatment of an inflammatory disorder, where the treatment comprises administration of sex-matched stem or progenitor cells, such as mesenchymal stern cells (MSCs), or sex-matched cell secretions, or a combination thereof to a subject. The invention also relates to kits and compositions which may be used in such methods.

### BACKGROUND

Mesenchymal stern cells (MSCs) are regarded as being immune-privileged and it is widely believed that for allogeneic treatments immunological matching of donors to patients is not required.

MSCs lack expression of major histocompatibility complex (MHC) class II surface molecules and have specific immune-suppressive properties that are believed to allow the cells to avoid entirely the donor immune responses, which normally result in primary rejection of allogeneic tissues.

At present, there are a significant number of clinical trials exploring the use of MSCs for the treatment of various diseases, including osteoarthritis, myocardial infarction, stroke, and others with clear involvement of the immune system, such as graft-versus-host disease, Crohn's disease, rheumatoid arthritis and diabetes. MSCs are being used as cell therapy to treat defects in bone and cartilage and to help in wound healing, or in combination with biomaterials in tissue engineering development.

Although these studies and related research have shown promising results, there is still a need for improved methods for the use of MSCs for the treatment of various diseases, including inflammatory conditions.

### SUMMARY OF INVENTION

The inventors have surprisingly discovered that there is an improved therapeutic effect from allogeneic MSCs when the sex of the donor and the patient are matched.

In a first aspect of the invention there is provided a method for treatment of an inflammatory disorder in a subject, the method comprising the steps of: identifying the sex of the subject; selecting a pharmaceutical composition comprising stem or progenitor cells derived from one or more individuals of the same sex as the subject; and administering to said subject a therapeutically effective amount of the selected sex-matched cell composition.

In another aspect of the invention there is provided a method for treatment of an inflammatory disorder in a subject, the method comprising the steps of: identifying the sex of the subject; selecting a pharmaceutical composition comprising mesenchymal stem cells (MSCs) derived from one or more individuals of the same sex as the subject; and administering to said subject a therapeutically effective amount of the selected sex-matched cell composition.

In another aspect of the invention there is provided a method for treatment of an inflammatory disorder in a subject, the method comprising the steps of: identifying the sex of the subject; selecting a pharmaceutical composition comprising cell secretions from cell culture of cells derived from one or more individuals of the same sex as the subject; and administering to said subject a therapeutically effective amount of the selected sex-matched cell secretions composition. In an embodiment the cell secretions are derived from cell culture of stem or progenitor cells described herein. In an embodiment the cell secretions are derived from cell culture of MSCs described herein. In an embodiment the cell secretions are derived from adipose tissue-derived cells, such as cells of the stromal vascular fraction or adipocytes.

In a further aspect of the invention there is provided a method for treatment of osteoarthritis in a subject, the method comprising the steps of: identifying the sex of the subject; selecting a pharmaceutical composition comprising mesenchymal stem cells (MSCs) derived from one or more individuals of the same sex as the subject; and administering to said subject a therapeutically effective amount of the selected sex-matched cell composition.

In an embodiment the cell secretions are from culture of cells derived from one or more individuals of the same species as the recipient subject. In an embodiment the cell secretions are from culture of cells derived from one or more individuals of a different species as the recipient subject. Hence the cell secretions may be described as xenogeneic to the recipient subject.

In an embodiment the inflammatory disorder is selected from a joint-related inflammatory disorder, corneal inflammation, skin inflammation or inflammation associated with wounding. In certain embodiments the inflammatory disorder is a skin or corneal ulcer. In an embodiment the inflammatory disorder is arthritis. In an embodiment the inflammatory disorder is osteoarthritis.

In an embodiment the subject is a female. In an embodiment the subject is a male. In an embodiment the methods of the invention are non-autologous methods.

In an embodiment the MSCs are allogeneic to the subject.

In an embodiment the pharmaceutical composition comprising mesenchymal stem cells (MSCs) is a homogeneous composition of MSCs. In an embodiment the MSCs are culture expanded cells. In an embodiment the pharmaceutical composition comprising mesenchymal stem cells (MSCs) further comprises secretions. In an embodiment the subject is administered a therapeutically effective amount of a pharmaceutical composition comprising cell secretions. In an embodiment the cell secretions are derived from cell culture of cells derived from one or more individuals of the same sex as the recipient subject. In an embodiment the cell secretions and the mesenchymal stem cells (MSCs) are together in one pharmaceutical composition.

In an embodiment the MSCs are derived from adipose tissue, bone marrow, placenta, blood, or cord blood. In an embodiment the MSCs are derived from multiple donor animals of the same species and sex.

In an embodiment the MSCs are derived from a de-sexed donor animal. In an embodiment the de-sexed donor animal is a female dog.

In an embodiment the subject is a human subject.

In an embodiment the subject is a non-human animal. In an embodiment the non-human subject is selected from the group consisting of ovine, bovine, equine, porcine, feline, canine, primate, and rodent. In an embodiment the non-human subject is a dog.

In an embodiment the subject is a de-sexed non-human female animal. In an embodiment the subject is a de-sexed non-human male animal.

In a further aspect of the invention there is provided a kit comprising, in separate containers, (i) a pharmaceutical composition comprising stem or progenitor cells derived from a female donor animal, and (ii) a pharmaceutical composition comprising stern or progenitor cells derived from a male donor animal.

In a further aspect of the invention there is provided a kit comprising, in separate containers, (i) a pharmaceutical composition comprising mesenchymal stern cells (MSCs) derived from a female donor animal, and (ii) a pharmaceutical composition comprising mesenchymal stern cells (MSCs) derived from a male donor animal.

In a further aspect of the invention there is provided a kit comprising, in separate containers, (i) a pharmaceutical composition comprising cell secretions from cell culture of cells derived from one or more female donor animals, and (ii) a pharmaceutical composition comprising cell secretions from cell culture of cells derived from one or more male donor animals. In an embodiment the cell secretions are derived from cell culture of stem or progenitor cells described herein. In an embodiment the cell secretions are derived from cell culture of MSCs described herein. In an embodiment the cell secretions are derived from adipose tissue-derived cells, such as cells of the stromal vascular fraction or adipocytes.

In an embodiment the kit further comprises instructions for sex-matched administration of a pharmaceutical composition comprising MSCs or of a pharmaceutical composition comprising stem or progenitor cells or of a pharmaceutical composition comprising cell secretions from cell culture of such cells, to a subject having an inflammatory disease.

In an embodiment the kit further comprises instructions for sex-matched administration of a pharmaceutical composition comprising MSCs to a subject having osteoarthritis.

In an embodiment the pharmaceutical composition is a cryopreserved pharmaceutical composition of stem or progenitor cells.

In an embodiment the pharmaceutical composition is a cryopreserved pharmaceutical composition of MSCs.

In an embodiment the pharmaceutical composition comprising mesenchymal stem cells (MSCs) is a homogeneous composition of MSCs. In an embodiment the MSCs are culture expanded cells.

In an embodiment the MSCs are derived from adipose tissue, bone marrow, placenta, blood, or cord blood. In an embodiment the MSCs derived from a female donor animal and the MSCs derived from a male donor animal are animals of the same species. In an embodiment the MSCs are derived from multiple donor animals of the same species and sex.

In an embodiment the MSCs and/or the stem or progenitor cells and/or the cell secretions are derived from a de-sexed donor animal. In an embodiment the de-sexed donor animal is a female dog.

In an embodiment the donor animal is a human. In an embodiment the donor animal is a non-human animal. In an embodiment the non-human donor animal is selected from the group consisting of ovine, bovine, equine, porcine, feline, canine, primate, and rodent. In an embodiment the non-human donor animal is a dog.

In an embodiment one or more of the pharmaceutical composition(s) comprising mesenchymal stem cells (MSCs) or comprising stern or progenitor cells further comprises secretions. In an embodiment the kit further comprises a pharmaceutical composition comprising cell secretions. In an embodiment the cell secretions are derived from cell culture of cells derived from one or more individuals of the same sex as the recipient subject. In an embodiment the cell secretions and the mesenchymal stem cells (MSCs) are together in one pharmaceutical composition. In an embodiment the pharmaceutical composition comprises human MSCs and secretions. In an embodiment the cell secretions and the mesenchymal stern cells (MSCs) together in one pharmaceutical composition are derived from one or more donors of the same sex.

In a further aspect of the invention there is provided a method for the treatment of pain associated with an inflammatory disorder in a subject, the method comprising the steps of: identifying the sex of the subject; selecting a pharmaceutical composition comprising stem or progenitor cells derived from one or more individuals of the same sex as the subject; and administering to said subject a therapeutically effective amount of the selected sex-matched cell composition.

In another aspect of the invention there is provided a method for treatment of pain associated with an inflammatory disorder in a subject, the method comprising the steps of: identifying the sex of the subject; selecting a pharmaceutical composition comprising cell secretions from cell culture of cells derived from one or more individuals of the same sex as the subject; and administering to said subject a therapeutically effective amount of the selected sex-matched cell secretions composition. In an embodiment the cell secretions are derived from cell culture of stem or progenitor cells described herein. In an embodiment the cell secretions are derived from cell culture of MSCs described herein. In an embodiment the cell secretions are derived from adipose tissue-derived cells, such as cells of the stromal vascular fraction or adipocytes.

In a further aspect of the invention there is provided a method for the treatment of pain associated with osteoarthritis in a subject, the method comprising the steps of: identifying the sex of the subject; selecting a pharmaceutical composition comprising mesenchymal stem cells (MSCs) derived from one or more individuals of the same sex as the subject; and administering to said subject a therapeutically effective amount of the selected sex-matched cell composition.

In a further aspect of the invention there is provided a method for reducing an adverse immune response in a subject administered sex mismatched stem or progenitor cells or cell secretions from culture of such cells, the method comprising (i) administering to said subject (i) one or more immune suppressant drug(s) and or (ii) conditioned media from cell culture of stem cells. In an embodiment the subject is a male animal. In an embodiment the one or more immune suppressant drug(s) is administered to the subject prior to administration of the sex mismatched stem or progenitor cells or cell secretions from culture of such cells, In an embodiment the subject has an inflammatory disorder, such as osteoarthritis. In an embodiment the method is a non-autologous method.

In a further aspect of the invention there is provided a method for reducing an adverse immune response in a subject administered sex mismatched MSCs, the method comprising (i) administering to said subject (i) one or more immune suppressant drug(s) and or (ii) conditioned media from cell culture of stem cells. In an embodiment the subject is a male animal. In an embodiment the one or more immune suppressant drug(s) is administered to the subject prior to administration of the MSCs. In an embodiment the subject has osteoarthritis. In an embodiment the method is a non-autologous method.

The summary of the invention described above is not limiting and other features and advantages of the invention will be apparent from the following detailed description of the preferred embodiments, as well as from the claims.

### BRIEF DESCRIPTION OF DRAWINGS

FIG. 1. Mean reduction of Pain Severity Score (PSS) from baseline for 5 male and 5 female dogs treated with female cells. The Y-axis represents the change in pain compared to pre-treatment, with a negative score being an improvement and a positive score being a worsening of pain. The X-axis represents the time since treatment. The male dogs showed a marginal improvement at day 10 and were worse than pre-treatment at 1 month and 2 months. The female dogs showed an improvement at day 10 and at 1 month and 2 months.

FIG. 2. Mean reduction of Pain Interference Score (PIS) from baseline for 5 male and 5 female dogs treated with female cells. The Y-axis represents the change in pain compared to pre-treatment, with a negative score being an improvement and a positive score being a worsening of pain. The X-axis represents the time since treatment. The male dogs showed an improvement at day 10 and were worse than pre-treatment at 1 month and marginally improved at 2 months. The female dogs showed a large improvement at day 10 and a very large improvement at 1 month and 2 months.

FIG. 3. Mean reduction of Pain Severity Score from baseline for dogs treated with female cells in an open trial. The Y-axis represents the change in pain compared to pre-treatment, with a negative score being an improvement and a positive score being a worsening of pain. The X-axis represents the time since treatment. The female dogs showed a much larger improvement than the male dogs at all time points.

FIG. 4. Mean reduction of Pain Interference Score from baseline for dogs treated with female cells in an open trial. The Y-axis represents the change in pain compared to pre-treatment, with a negative score being an improvement and a positive score being a worsening of pain. The X-axis represents the time since treatment. The female dogs showed a larger improvement than the male dogs at all time points.

FIG. 5. Mean reduction of Pain Severity Score from baseline for 25 dogs treated with female cells. The Y-axis represents the change in pain compared to pre-treatment, with a negative score being an improvement and a positive score being a worsening of pain. The X-axis represents the time since treatment. The female dogs showed a much larger improvement than the male dogs at all time points.

FIG. 6. Mean reduction of Pain Inference Score from baseline for 25 dogs treated with female cells. The Y-axis represents the change in pain compared to pre-treatment, with a negative score being an improvement and a positive score being a worsening of pain. The X-axis represents the time since treatment. The female dogs showed a much larger improvement than the male dogs at all time points.

FIG. 7. The percentage of dogs in the open trial that improved PSS and PIS by greater than 1 and by greater than 2. More female dogs improved than male dogs.

FIG. 8. Mean PSS scores from dogs treated with male cells. Male dogs responded better than female dogs.

FIG. 9. Mean PIS scores from dogs treated with male cells. Male dogs responded better than female dogs.

FIG. 10. White blood cell (WBC) counts in the synovial fluid of horses given weekly injections of female MSCs.

FIG. 11. Analysis of the antibody response to female MSCs in 3 horses, after five intra-articular injections of female MSCs. The histograms represent the average fluorescent intensity of cells from 3 replicate analysis of 2000 cells for each horse pre-injection (light shading) and post-injection (darker shading). The error bars represent the standard deviation.
FIG. 12. Analysis of antibody response to male (darker shading) and female MSCs (lighter shading) in horses injected five times with female MSCs. The histograms represent the average fold-increase in antibody response from three replicates for each horse serum. The error bars represent the standard deviation.

### DESCRIPTION OF EMBODIMENTS

Throughout this specification, reference to “a” or “one” element does not exclude the plural, unless context determines otherwise. Similarly, reference to “an embodiment” does not exclude the characteristic of that described embodiment applying in combination with one or more other embodiments described, unless the context determines otherwise.

The term “therapeutically effective amount” as used herein includes within its meaning a non-toxic but sufficient amount of a compound or composition for use in the invention to provide the desired therapeutic effect. The exact amount required will vary from subject to subject depending on factors such as the species being treated, the age and general condition of the subject, co-morbidities, the severity of the condition being treated, the particular agent being administered and the mode of administration and so forth. Thus, for any given case, an appropriate “effective amount” may be determined by one of ordinary skill in the art using only routine methods.

In the context of this specification, the term “comprising” means including, but not necessarily solely including. Furthermore, variations of the word “comprising”, such as “comprise” and “comprises”, have correspondingly varied meanings. Hence, the term “comprising” and variations thereof is used in an inclusive rather than exclusive meaning such that additional integers or features may optionally be present in a composition, method, etc. that is described as comprising integer A, or comprising integer A and B, etc.

In the context of this specification the terms “about” and “approximately” will be understood as indicating the usual tolerances that a skilled addressee would associate with the given value.

In the context of this specification, where a range is stated for a parameter it will be understood that the parameter includes all values within the stated range, inclusive of the stated endpoints of the range. For example, a range of “5 to 10” will be understood to include the values 5, 6, 7, 8, 9, and 10 as well as any sub-range within the stated range, such as to include the sub-range of 6 to 10, 7 to 10, 6 to 9, 7 to 9, etc., and inclusive of any value and range between the integers which is reasonable in the context of the range stated, such as 5.5, 6.5, 7.5, 5.5 to 8.5 and 6.5 to 9, etc.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention before the priority date of this application.

In the context of this specification, the terms “plurality” and “multiple” mean any number greater than one.

It is to be noted that reference herein to use of the inventive methods and compositions in treatment or therapy will be understood to be applicable to human and non-human, such as veterinary, applications. Hence it will be understood that, except where otherwise indicated, reference to a patient, subject or individual means a human or a non-human, such as an individual of any species of social, economic, agricultural or research importance including but not limited to members of the classifications of ovine, bovine, equine, porcine, feline, canine, primates, rodents, especially domesticated or farmed members of those classifications, such as sheep, cattle, horses, pigs and dogs.

Where examples of various embodiments or aspects of the invention are described herein they will generally be prefaced by appropriate terms including “such as” or “for example”, or “including”. It will be understood that the examples are being described as inclusive possibilities, such as for the purpose of illustration or understanding and are not, unless the context indicates otherwise, being provided as limiting.

The pharmaceutical composition referred to herein may also be referred to as a medicament, such as when intended for therapeutic use. Hence, it will be understood that where the invention is described as including the use of a composition of described components for the preparation of a pharmaceutical composition for an intended therapeutic purpose, that description equally means use for the preparation of a medicament for that intended therapeutic purpose, unless the context indicates otherwise.

To the extent that it is permitted, all references cited herein are incorporated by reference in their entirety.

The invention described herein relates to improved methods for treatment of an inflammatory disorder and or the alleviation of pain associated therewith in a subject. As noted herein the inventors have surprisingly identified that there is an improved therapeutic effect from allogeneic MSCs when the sex of the donor and the patient or subject being treated are matched. This has permitted the inventors to devise an improved method for treating an inflammatory disorder, exemplified by osteoarthritis, in a subject, the method comprising the steps of: identifying the sex of the subject; selecting a pharmaceutical composition comprising stem or progenitor cells, exemplified by mesenchymal stem cells (MSCs), or sex-matched cell secretions, or a combination thereof, derived from one or more individuals of the same sex as the subject; and administering to said subject a therapeutically effective amount of the selected sex-matched composition. For example, where the subject being treated is female, the pharmaceutical composition used in the treatment will comprise stem or progenitor cells, e.g. MSCs, or sex-matched cell secretions, or a combination thereof, derived from a female donor. Alternatively, where the subject being treated is male, the pharmaceutical composition used in the treatment will comprise stem or progenitor cells, e.g. MSCs, or sex-matched cell secretions, or a combination thereof, derived from a male donor.

It will be understood that the improved therapies described herein, arising from the observation that a recipient subject being treated for osteoarthritis or pain associated therewith when treated with MSCs derived from a sex-matched source, are anticipated to occur notwithstanding the fact that the pharmaceutical composition administered to the subject may also contain some cells not derived from a donor of the same sex as the recipient subject. For example, in the treatment of a female subject suffering from an inflammatory disorder, administration of a pharmaceutical composition comprising MSCs derived from one or more females but which composition contains a minor proportion of MSCs derived from one or more male sources would be expected to be therapeutically beneficial to the subject. Hence, a pharmaceutical composition comprising mesenchymal stem cells (MSCs) derived from one or more individuals of the same sex as the recipient subject may also contain a minor proportion of cells derived from the opposite sex. Preferably, a minor proportion, when present, would be less than about 30%, or less than about 20%, or less than about 15%, or less than about 10%, or less than about 5%, or less than about 2% of the total content of cells, such as the total number of cells, in the administered composition. This applies in a corresponding manner where the composition comprises cell secretions, such that a minor proportion of cell secretions not derived from a sex-matched source may be present in the composition. The scope of the invention for the purposes of infringement will thus not be avoided by the inclusion of a minor proportion of non-sex matched material. Similarly, where the methods of the invention are described or claimed as being non-autologous methods, the scope of the invention for the purposes of infringement will thus not be avoided by the inclusion of a minor proportion of cells or cell secretions autologous to the subject individual being treated.

As demonstrated herein the inventors have also found that there is an immune response to sex mismatched MSCs. This finding has implications for all types of diseases and conditions that can be treated with allogeneic stem cells. The immune response will cause cells to be attacked and destroyed by the recipient subject's immune system. This may result in inflammation at the site of injection or systemic inflammation. It will also increase the likelihood of cells not embedding and as a result will prevent or diminish the capability for regeneration of damaged tissue.

The examples herein demonstrate that an immune response to allogeneic stem cells can be prevented by immunizing with sex-matched cells.

The immune response arising from the use of sex mismatched cells, such as that observed when male animals undergoing treatment are administered female donor MSCs, can be blocked or prevented by treating with immune suppressant drugs such as steroids or by administering conditioned media from stem cells along with the stem cells. Whilst that finding is beneficial, for example in situations where sex-matched cells are not available, the instant invention also offers a desirable alternative. That is, by administering sex-matched MSCs to a subject, the need for an additional drug burden of immune suppressant drugs on the individual is avoided as is the need for administration of immunosuppressive conditioned media in addition to the cells. The instant invention thus offers a simple alternative to avoiding or reducing the detrimental effects of an immune response to allogeneic stem cells in a subject undergoing MSC-based treatment.

### Inflammatory Disorders

The methods and compositions described herein may be used for the treatment of an inflammatory disorder and/or for alleviating pain associated with an inflammatory disorder in a subject. Inflammation may arise as a response to an injury or abnormal stimulation caused by a physical, chemical, or biologic agent. An inflammation reaction may include the local reactions and resulting morphologic changes, destruction or removal of injurious material such as an infective organism, and responses that lead to repair and healing. The term “inflammatory” when used in reference to a disorder refers to a pathological process which is caused by, resulting from, or resulting in inflammation that is inappropriate or which does not resolve in the normal manner Inflammatory disorders may be systemic or localized to particular tissues or organs.

Inflammation is known to occur in many disorders which include, but are not limited to: Systemic Inflammatory Response (SIRS); Alzheimer's Disease (and associated conditions and symptoms including: chronic neuroinflammation, glial activation; increased microglia; neuritic plaque formation; Parkinson's disease; Amyotrophic Lateral Sclerosis (ALS), arthritis (and associated conditions and symptoms including, but not limited to: acute joint inflammation, antigen-induced arthritis, arthritis associated with chronic lymphocytic thyroiditis, collagen-induced arthritis, juvenile arthritis, rheumatoid arthritis, osteoarthritis, prognosis and streptococcus-induced arthritis, spondyloarthropathies, and gouty arthritis), asthma (and associated conditions and symptoms, including: bronchial asthma; chronic obstructive airway disease, chronic obstructive pulmonary disease, juvenile asthma and occupational asthma); ischemic stroke; traumatic brain injury (TBI); neonatal hypoxic ischemia; cardiovascular diseases (and associated conditions and symptoms, including atherosclerosis, autoimmune myocarditis, acute myocardial infarction, peripheral vascular disease, chronic cardiac hypoxia, congestive heart failure, coronary artery disease, cardiomyopathy and cardiac cell dysfunction, including: aortic smooth muscle cell activation, cardiac cell apoptosis and immunomodulation of cardiac cell function); diabetes (and associated conditions, including autoimmune diabetes, insulin-dependent (Type 1) diabetes, diabetic periodontitis, diabetic retinopathy, and diabetic nephropathy); gastrointestinal inflammations (and related conditions and symptoms, including celiac disease, associated osteopenia, chronic colitis, Crohn's disease, inflammatory bowel disease and ulcerative colitis); gastric ulcers; hepatic inflammations such as viral and other types of hepatitis, cholesterol gallstones and hepatic fibrosis; HIV infection (and associated conditions, including degenerative responses, neurodegenerative responses, and HIV associated Hodgkin's Disease); Kawasaki's Syndrome (and associated diseases and conditions, including mucocutaneous lymph node syndrome, cervical lymphadenopathy, coronary artery lesions, edema, fever, increased leukocytes, mild anemia, skin peeling, rash, conjunctiva redness, thrombocytosis); kidney disease and nephropathies (and associated diseases and conditions, including diabetic nephropathy, end stage renal disease, acute and chronic glomerulonephritis, acute and chronic interstitial nephritis, lupus nephritis, Goodpasture's syndrome, hemodialysis survival and renal ischemic reperfusion injury); neurodegenerative diseases or neuropathological conditions (and associated diseases and conditions, including acute neurodegeneration, induction of IL-I in aging and neurodegenerative disease, IL-I induced plasticity of hypothalamic neurons and chronic stress hyperresponsiveness, myelopathy); dry eye, ophthalmopathies (and associated diseases and conditions, including diabetic retinopathy, Graves' ophthalmopathy, inflammation associated with corneal injury or infection including corneal ulceration, and uveitis), osteoporosis (and associated diseases and conditions, including alveolar, femoral, radial, vertebral or wrist bone loss or fracture incidence, postmenopausal bone loss, fracture incidence or rate of bone loss); otitis media (adult or paediatric); pancreatitis or pancreatic acinitis; periodontal disease (and associated diseases and conditions, including adult, early onset and diabetic); endometritis and endometriosis; spinal conditions including orthopaedic conditions of the spine, back pain, spinal fusion, spinal cord injury, intervertebral disc repair; pulmonary diseases, including chronic lung disease, chronic sinusitis, hyaline membrane disease, hypoxia and pulmonary disease in SIDS; restenosis of coronary or other vascular grafts; rheumatism including rheumatoid arthritis, rheumatic Aschoff bodies, rheumatic diseases and rheumatic myocarditis; thyroiditis including chronic lymphocytic thyroiditis; urinary tract infections including chronic prostatitis, chronic pelvic pain syndrome and urolithiasis; immunological disorders, including autoimmune diseases, such as alopecia aerata, autoimmune myocarditis, Graves' disease, Graves ophthalmopathy, lichen sclerosis, multiple sclerosis, psoriasis, systemic lupus erythematosus, systemic sclerosis, thyroid diseases (e.g. goitre and struma lymphomatosa (Hashimoto's thyroiditis, lymphadenoid goitre); bone marrow transplantation; organ transplantation; graft versus host disease; lung injury (acute hemorrhagic lung injury, Goodpasture's syndrome, acute ischemic reperfusion), myocardial dysfunction, caused by occupational and environmental pollutants (e.g. susceptibility to toxic oil syndrome silicosis), radiation trauma, and efficiency of wound healing responses (e.g. burn or thermal wounds, chronic wounds, surgical wounds and spinal cord injuries), septicaemia, acute phase response (e.g. febrile response), general inflammatory response, acute respiratory distress response, acute systemic inflammatory response, skin disorders (e.g. psoriasis, acne, acne rosacea, acne vulgaris, eczema, cellulitis, post hepatic neuralgia, neuropathic pain, dermatitis, atopic dermatitis, nappy rash, scar reduction associated with an inflammatory skin condition; burns, wound healing, bed sores, ulcers), adhesion, immuno-inflammatory response, neuroendocrine response, fever development and resistance, acute-phase response, stress response, disease susceptibility, repetitive motion stress, tennis elbow, and pain management and response.

In particular embodiments the inflammatory disorder is selected from joint-related inflammatory disorders, corneal inflammation, skin inflammation or wound healing.

In particular embodiments the joint-related inflammatory disorder is arthritis, such as osteoarthritis.

### Osteoarthritis

Osteoarthritis (OA) is an idiopathic, incurable chronic and debilitating musculoskeletal disease and is reported by more than 1.4 million people in Australia. OA onset is most closely associated with ageing and the key observations are cartilage changes and pain. It is classically referred to as a non-inflammatory disease but it is increasingly evident that inflammation plays a major role in OA disease progression. Patients with OA are typically managed with non-steroidal anti-inflammatory drugs (NSAIDs) and analgesics to alleviate OA symptoms and to control the pain in affected joints. Currently, when NSAIDs and also corticosteroid therapy are no longer beneficial, the usual treatment is total joint arthroplasty. This poses a significant problem for patients who are 30-60 years old. Many orthopaedic surgeons are hesitant to perform a joint replacement on people under 50 because the implant is unlikely to last their lifetime.

In recent years there has been a shift in medical research towards innovative regenerative treatments for a variety of diseases. In joint diseases such as arthritis, a number of research groups have used animal models of OA to explore the use of adult mesenchymal stem cells (MSCs) as a potential regenerative therapy. In animal models of acute and chronic cartilage damage, treatment with MSCs produces meniscal and hyaline cartilage regeneration and reductions in OA-like disease progression, cartilage loss, osteophyte formation and subchondral thickening. These cells have also been demonstrated to have significant anti-inflammatory and immunomodulatory effects through the secretion of bioactive factors.

As described herein the present invention provides improved cell-based therapeutic methods for treatment of osteoarthritis in a subject, by administering sex-matched stem or progenitor cells, for example mesenchymal stem cells (MSCs), to the subject. The terms “treating”, “treatment”, “therapy” and the like in the context of the present specification refer to the alleviation of the symptoms and/or the underlying cause of the condition or disease, such as osteoarthritis. In certain embodiments a treatment will slow, delay or halt the progression of a disorder or the symptoms of the disorder or injury, or reverse the progression of the disorder or injury, at least temporarily. Hence, in the context of this invention the word “treatment” or derivations thereof such as “treating” when used in relation to a therapeutic application includes all aspects of a therapy, such as the alleviation of pain associated with the condition being treated, alleviation of the severity of the condition being treated, improvement in one or more symptoms of the condition being treated, etc. Use of the word “treatment” or derivatives thereof will be understood to mean that the subject being “treated” may experience any one or more of the aforementioned benefits.

It will be understood that the methods of the invention may also benefit the subject through alleviation of pain associated with the inflammatory condition, such as pain associated with osteoarthritis. The term “alleviation of pain associated with an inflammatory condition” is intended to encompass a reduction in pain which results from the subject's condition, but not necessarily treating the underlying condition which causes the pain.

Typically, in working the methods of the invention, the treating physician, for example, a doctor, a veterinarian, or nurse, would have available to them a pharmaceutical composition comprising stem or progenitor cells, such as MSCs, derived from a female donor or cell secretions from culture of such cells and a separate pharmaceutical composition comprising stem or progenitor cells, such as MSCs, derived from a male donor secretions from culture of such cells. The donor animal is typically an animal of the same species as the subject being treated, such that the stem or progenitor cells, eg the MSCs, or secretions of cultures thereof, used in the treatment are allogeneic stem or progenitor cells, eg MSCs, or secretions of cultures of said allogeneic cells.

Non-Inflammatory disorders

The methods and compositions described herein may be used for the treatment of a non-inflammatory disorder and/or alleviating pain associated with such a disorder. The non-inflammatory disorder is associated with one or more of the following conditions in a subject: dry skin, itchy skin, insect bite or sting, sun burn, wrinkling of the skin, thin skin, cracking of the skin, acne, scarring, stretch marks, sun spots, age spots, liver spots, puffiness and or dark circles around the eyes, athlete's foot, warts, surgery-related hair loss, chemotherapy-related hair loss, radiation exposure-related hair loss, alopecia, male pattern baldness or female pattern baldness.

### Stem and Progenitor Cells

The stem or progenitor cells are derived from one or more individuals of the same sex as the subject being, or intended to be, treated.

As used herein, the term “stem cell” refers to a cell that is totipotent or pluripotent or multipotent and is capable of differentiating into one or more different cell types, such as embryonic stems cells, embryonic germline cells, mesenchymoangioblasts, stem cells isolated from adult tissue, for example adipose tissue, neural tissue (e.g. brain or olfactory mucosa), bone marrow, placenta, blood, or cord blood etc.

As used herein, the term “progenitor cell” refers to a partially differentiated cell or an undifferentiated cell, e.g. derived from a stem cell, and is not itself a stem cell. Some progenitor cells can produce progeny that are capable of differentiating into more than one cell type.

The stem or progenitor cells derived from a sex-matched individual include dedifferentiated cells. A dedifferentiated cell refers to a cell which has undergone a process wherein a more specialized cell having a more distinct form and function, and/or limited self-renewal and/or proliferative capacity becomes less specialized and acquires a greater self-renewal and/or proliferative capacity or differentiation capacity (e.g. multipotent, pluripotent etc.). An induced Pluripotent Stem Cell (iPSC) is an example of a de-differentiated cell. iPSCs may be generated through a variety of methods. iPSCs may be generated via cellular reprogramming which occurs via the administration, or directed expression, of a combination of transcription factors or “reprogramming factors” including Oct4, Sox2, cMyc and Klf4. Methods for generating iPSCs from biological tissues are known and have been described in the art, for example, in Takahashi, K. & Yamanaka, S. (2006) Induction of pluripotent stem cells from mouse embryonic and adult fibroblast cultures by defined factors. Cell 126, 663-676.

In this context the stem or progenitor cells being “derived from” simply means that the stem cells, or more typically parental cells from which the stem or progenitor cells used for the administration to a subject, were obtained or sourced from a donor animal of the same sex as that to which the cells are administered.

Mesenchymal Stem Cells (MSCs)

The MSCs are derived from one or more individuals of the same sex as the subject being, or intended to be, treated. In this context the MSCs being “derived from” simply means that the MSCs, or more typically parental cells from which the MSCs used for the administration to a subject, were obtained or sourced from a donor animal of the same sex as that to which the MSCs are administered. To provide one illustration, the examples herein describe administration of cryopreserved MSCs, those MSCs having been prepared by tissue culture of a cell suspension comprising MSCs, the cell suspension used in the tissue culture having been prepared from a sample of adipose tissue which was obtained initially from a male dog or from a female dog. Where the ultimate source was a female, the MSCs may be said to be derived from a female donor and where the ultimate source was a male, the MSCs may be said to be derived from a male donor.

Mesenchymal stem cells (MSCs) are post-natal, multipotent, adult stem cells. Mesenchymal stem cells (MSCs) are present in many tissues in the body and play an important role in tissue repair and regeneration. For therapeutic purposes MSCs are commonly harvested from bone marrow, placenta, cord blood and adipose tissue In many circumstances the cells are expanded by tissue culture prior to use.

The mesenchymal stem cells (MSCs) may originate from any tissue where MSCs are found, including, but not limited to, bone marrow, skeletal muscle, skin, connective tissue, and adipose tissue, placenta, blood or cord blood. By originate is meant the tissue type that the MSCs are isolated from for use in the methods or compositions of the present invention. In a particular embodiment, the MCSs may originate from bone marrow or adipose tissue. The MSCs may be isolated from a tissue specifically for the purposes of the methods and compositions of the invention, or the MSCs may have previously been isolated from a tissue source in a procedure unrelated to the methods or compositions of the invention. The isolation of MSCs from suitable tissue or the preparation of a composition comprising MSCs may or may not constitute a step or steps of performance of the method of the invention.

Adipose tissue has the unique advantage as a source of MSCs that such large numbers of MSCs are present in the tissue, containing for example 500-1000 times more MSCs per gram than bone marrow, that for many applications the cells do not need to be expanded by tissue culture. Along with an abundance of MSCs, adipose tissue also comprises immune cells, vascular smooth muscle cells, endothelial cells, and pericytes, which collectively are termed the stromal vascular fraction (SVF). The ability to obtain large quantities of adipose tissue through standard liposuction techniques and the ability to rapidly isolate the SVF, from which MSCs for use in the invention may be generated, either with or without culture expansion. Regardless of the original source tissue of the MSCs, MSCs used in the methods of the invention may or may not be culture expanded cells. Typically, MSCs used in the methods of the invention are culture expanded cells.

Compositions comprising MSCs may comprise MSCs initially isolated from a biological sample comprising tissue where MSCs are found, such as described above. The MSCs may be isolated from a biological sample, and then handled, maintained and stored, according to appropriate methods known to those skilled in the art. It would be understood that appropriate methods of isolation, handling, maintenance and storage would be methods that are conducive to the MSCs retaining multipotency. The MSCs may, for example, be used in the method of the present invention immediately after being isolated from a biological sample. Alternatively, the MSCs may go through one or more stages of freezing, and/or passaging in cell culture prior to use. For example, the MSCs isolated from a biological sample may be passaged in cell culture once prior to use in the method, or the MSCs may be isolated from a biological sample and then frozen and thawed prior to use, or the isolated MSCs may be frozen, thawed and then passaged once in cell culture prior to use. The MSCs may, for example, be isolated from a biological sample and passaged in cell culture, then frozen and thawed, and then passaged one or more times in cell culture prior to use. In another example, the MCSs may be isolated from a biological sample and passaged one or more times in cell culture prior to use. It would be understood that passaging involves growing of the MSCs in cell culture media, and is often referred to as expanding, colony expansion, splitting.

Methods for isolating MSCs from biological tissues are known in the art as are methods for in vitro culturing of MSCs are known in the art and have been described in the art, for example, in Gimble, J., Katz, A., & Bunnell, B. (2007). Adipose-derived stem cells for regenerative medicine. Circ Res, 100(9), 1249-1260. doi:100/9/1249 [pii]10.1161/01.RES.0000265074.83288.09; Soleimani, M., & Nadri, S. (2009). A protocol for isolation and culture of mesenchymal stem cells from mouse bone marrow. Nature Protocols, 4(1), 102-106. doi:10.1038/nprot.2008.221.

It would be understood that methods for the isolation of MSCs from a biological sample may not produce a sample that is comprised of only MSCs. The compositions comprising MSCs may comprise cells that are not MSCs, as well as non-cellular components. These non-cellular components and non-MSCs may, for example, have originated from the biological sample that the MSCs were isolated from, or they may, for example, be from buffers, solutions or media used during handling, maintenance, culturing and storage of the MSCs. The cells that are not MSCs may, for example, be from connective tissue, blood, bone marrow, adipose tissue, blood vessels, nervous tissue, muscle tissue and/or stromal tissue. The cells may be adipocytes that may have, for example, been in the biological sample that the MSCs were isolated from. In certain embodiments, the composition comprising the MSCs further comprises adipocytes. The non-cellular components may be, for example, tissue fluid, cell culture media, plasma components, extra-cellular matrix, enzymes, growth factors and cytokines. The non-cellular components may, for example, be components of the serum used during the passaging of the MSCs.

Typically, MSCs used in the methods of the invention are culture expanded, for example as culture expansion provides greater control over the uniformity of the cell composition, typically in that a culture expanded composition of MSCs will contain little or no non-MSC cells. Culture expanded MSCs are typically also available to the practitioner in greater numbers than MSCs directly obtained from a biological sample without culture expansion.

### Pharmaceutical Compositions

The pharmaceutical compositions for use in the methods of the invention comprise sex-matched stem or progenitor cells, such as MSCs, in a pharmaceutically acceptable carrier, excipient or adjuvant. The compositions for use in the methods of the invention are typically “pharmaceutically acceptable”. The term “pharmaceutically acceptable” as used herein in the context of various components relevant to the invention, such as carriers, diluents, cryopreservatives, is intended to encompass not only such components which are suitable for administration to a human subject, but also those suitable for administration to a non-human mammalian subject. In particular embodiments, the pharmaceutically acceptable component is suitable for administration to a non-human mammalian subject. In particular embodiments the pharmaceutically acceptable component is suitable for administration to a human subject. In particular embodiments, the pharmaceutically acceptable component is suitable for administration to a non-human mammalian subject and to a human subject.

A pharmaceutical composition comprising stem or progenitor cells or mesenchymal stem cells (MSCs) may additionally comprise components secreted from said cells, such as from said MSCs, such as cytokines secreted by the cells during cell culture. These may generally be referred to herein as cell secretions. The inclusion of such cell secretions in the composition may be beneficial in one or more of the cryopreservation of the MSCs prior to use in the method of the invention, or may assist in the preservation of the efficacy of the MSCs during retrieval from storage, or may assist in the additional therapeutic benefits to the subject, for example by the inclusion of beneficial cytokines, such as anti-inflammatory cytokines. WO2013/040649 entitled “Therapeutic methods and compositions” describes that the inclusion of cell secretions in a cell suspension comprising adipose-derived cells, such as cells of the stromal vascular fraction, are advantageous in the cryopreservation of the cells and in their efficacy when used for the treatment of inflammatory diseases such as osteoarthritis. As described in WO2013/040649, the entire contents of which are hereby incorporated by reference, secretions may be used with such cell compositions in a concentrated form, such as concentrated 2-fold, 5-fold, 10-fold or 20-fold. The inventors herein propose that the benefits of the inclusion of secretions demonstrated in WO2013/040649 for adipose tissue-derived mesenchymal stem cells will be applicable to mesenchymal stem cells obtained from other sources, such as described herein.

Cell secretions for administration to a subject may be supplied in a composition which also comprises the MSCs or other stem or progenitor cells or may be supplied as a separate pharmaceutical composition. As described herein the cell secretions may be derived from cell culture of cells derived from a donor of the same sex as the subject being treated, or may be derived from cell culture of cells derived from a donor of the opposite sex as the subject being treated. In a preferred embodiment, the cell secretions are derived from cell culture of cells derived from a donor of the same sex as the subject being treated. In an embodiment the cell secretions are derived from adipose tissue-derived cells, such as cells of the stromal vascular fraction or adipocytes. In an embodiment the cell secretions are from culture of cells derived from one or more individuals of the same species as the recipient subject. In an embodiment the cell secretions are from culture of cells derived from one or more individuals of a different species as the recipient subject.

The inventors herein demonstrate for the first time that a recipient subject may have an immune response to sex mismatched MSCs. This was particularly evident in the case of male subjects administered female donor MSCs. As demonstrated in WO2013/040649 cell secretions from stem cell culture can have an immune suppressant effect when administered to a subject. Arising from the finding herein that the use of sex-mismatched MSCs is associated with an immune response to the donor cells, the inventors propose the administration of cell secretions or conditioned media from stem cell culture where a subject is being treated with sex-mismatched MSCs. The cell secretions or conditioned media from stem cell culture may be administered to the subject in any appropriate manner to reduce or alleviate an adverse immune response by the recipient subject to the administered sex-mismatched MSCs. Typically, the cell secretions or conditioned media from stem cell culture would be administered to the subject prior to or at the same time as the sex-mismatched MSCs.

As an alternative to the use of cell secretions or conditioned media from stem cell culture to prevent or alleviate the adverse effects of an immune response of a recipient subject to sex-mismatched stem or progenitor cells or MSCs, the subject may be administered one or more immune suppressive drug(s). Immune suppressive drugs are known in the art and include for example steroids, such as dexamethasone. The drug(s) may be administered to the recipient subject in any appropriate manner, although typically will be administered prior to or with the sex-mismatched stem or progenitor cells or MSCs. The skilled addressee will be able to determine an appropriate dosage of said drug(s) to achieve the desired effect.

Administration of Sex-Matched Stem or Progenitor Cells and/or Cell Secretions to a Subject

Administration of the pharmaceutical composition may be by any appropriate means. Typically, in the situation where the subject is being treated for a joint related inflammatory condition or disease, such as arthritis, which may be osteoarthritis, the condition will affect one or more of the subject's joints. In an embodiment the method of treating the condition is by intra-articular injection into an affected joint. In certain embodiments the pharmaceutical composition comprising sex-matched stem or progenitor cells (e.g. MSCs) and/or cell secretions may be administered directly to the site of the inflammatory disorder, or to the site where the pain is experienced. For example, where the inflammatory disorder is a joint-related inflammatory disorder, the pharmaceutical composition may be administered directly into the synovial fluid and/or into or around the joint capsule, and/or into the musculature overlying or surrounding the joint, and/or subcutaneously to the tissue overlying the joint. In a preferred embodiment the joint-related inflammatory condition is osteoarthritis.

The quantity of pharmaceutical composition which may be administered will depend on the size and location of the joint, and the site of administration. Where administration is by injection into the synovial fluid of a joint, for example, the volume may be constrained by the volume of the synovial fluid which is held at the joint.

Typically joint-related inflammatory conditions in humans involve at least one joint in one or both hips, knees, ankles, elbows, shoulders, wrists, the metacarpo-phalangeal articulations or the phalangeal articulations, the metatarso-tarsal articulations or the tarsal articulations or between two or more vertebrae. For veterinary joint-related inflammatory disorders the corresponding joints are involved in mammalian animals, and these include the stifle and hock joints.

The pharmaceutical composition comprising sex-matched stem or progenitor cells (e.g. MSCs) and/or cell secretions may be administered to the subject patient at a site remote from the afflicted area. In this context, “remote” means that the administration is not direct application of the cell suspension to the site or joint most directly identified as being affected by the condition. Such methods are described for example in WO/2013/040649 entitled “Therapeutics using adipose cells and cell secretions”, the entire contents of which are incorporated herein by reference.

As an illustration, in the case of treatment of an arthritic joint, administration as previously described in the art involved injection of adipose tissue-derived cell suspensions directly into the afflicted joint. Such administration requires a high degree of skill on the part of the treating physician or clinician to ensure appropriate precision. The handling of the affected limb or joint required in such administration also increases the distress experienced by the patient, be they human or non-human.

For example, the remote administration may be by subcutaneous injection, such as in the scruff of the neck of an animal (for example a cat or dog) being treated, or by intramuscular injection. As a further example, administration to a dog by intramuscular injection may be in to thigh of the dog. As a further example, administration to a bovine by intramuscular injection may be in the caudal fold, the rump or the neck. As a further example, administration to an equine by intramuscular injection may be in the rump or the neck.

The method may comprise a single treatment of the subject or may comprise a course of treatment comprising a first dose and a second dose, or a first dose, a second dose and a third dose, or a first dose, a second dose, a third dose and a fourth dose, or a first dose, second dose, a third dose, a fourth dose and a fifth dose.

It will be understood that in the context of the methods of the invention a dose means the administration of the pharmaceutical composition comprising sex-matched stem or progenitor cells (e.g. MSCs) and/or cell secretions to the subject at a given time, whether that dose be administered in a single application or in more than one application. As an illustrative example, a dose may consist of a single administration, such as a single injection into a targeted site on the subject's body. As a further illustrative example, a dose may consist of multiple administrations to one or more targeted sites on the subject's body, such as multiple injections. Any of the first, and or subsequent doses, such as any of the second, third, fourth, fifth, etc., doses may therefore be administered as a single application or as multiple applications.

Any appropriate time period between the first and each subsequent dose may be used. It is notable that the methods of the invention do not require that the subject be experiencing a relapse of the condition or an increase of symptoms of the condition, such as might occur if a dose was becoming less effective, to qualify for or to be given a subsequent dose or doses. Instead, it is the intended course of treatment in the methods of the invention that the subject be administered multiple doses of the composition over a period of time for the treatment of the same condition in the individual over that time.

Where a subject undergoing treatment is to be administered stem or progenitor cells (e.g. MSCs) and/or cell secretions on multiple occasions, the decision as to when to administer a subsequent dose will typically be made by the individual who is supervising the subject's treatment, such as the treating physician, doctor, veterinarian or nurse. A combination of factors will typically be taken into account in making such a decision. For example, factors taken in to account may include a timescale of appropriateness as assessed on the basis of past experience, either with the individual subject patient's condition or others with similar conditions, or on the degree of debility of the subject, or on the degree of pain experienced by the subject or may be based on a test independent of the subject's own assessment.

For example, co-pending application PCT/AU2014/000951, entitled “Biomarkers for cell therapy”, describes methods for the use of biomarkers to assist a treating physician assess the progression of osteoarthritis and to assist in identifying appropriate treatment times for mesenchymal cell-based therapy. PCT/AU2014/000951 describes that macrophage migration inhibitory factor (MIF) is detectable in the serum of patients undergoing mesenchymal stem cell treatment for OA and that levels of detectable MIF correlate with treatment outcome, such as stabilisation or improvement. As exemplified therein in the treatment of OA, levels of detectable MIF correlate with treatment outcome, such as reduced cartilage degradation. MIF is an inflammatory cytokine that stimulates the degradation of damaged tissue.

PCT/AU2014/000951 also describes that CTX-II, a C-terminal telopeptide of type II collagen, is detectable in the serum and in the urine of patients undergoing treatment for OA and that levels of detectable CTX-II correlate with cartilage degradation. The serum levels of MIF correlate with reduced tissue degradation observed after MSC treatment, for example in OA, reduced serum MIF correlates with reduced urinary CTX II, which is a marker of cartilage degradation. Also described in PCT/AU2014/000951 COMP (cartilage oligomeric matrix protein) is an additional cartilage specific breakdown product that is well correlated with OA. It increases (in serum) during the progression of disease. As with CTX, PCT/AU2014/000951 demonstrates a post-treatment stabilisation or slight decrease of this marker.

Thus, the methods described in PCT/AU2014/000951, the entire contents of which are incorporated herein by reference, may be used by the treating physician to assist them in determining the progression of the OA in a subject patient, for example to assist in guiding decisions concerning an appropriate time at which to administer a therapeutic dose to the patient, for example a dose of sex-matched stem or progenitor cells (e.g. MSCs), to the patient. Advantageously such a method is independent of the patient's subjective assessment of their own condition such as self-reporting of pain scores or discomfort levels. The skilled addressee will appreciate that methods have also been described which would permit the physician to assess the progress of other inflammatory conditions in a subject.

In an embodiment a course of treatment comprises multiple doses in which each subsequent dose is separated in time from the previous dose by between one week and ten weeks. In an embodiment a course of treatment comprises multiple doses each subsequent dose separated in time from the previous dose by between two weeks and eight weeks. In an embodiment the course of treatment comprises multiple doses each subsequent dose separated in time from the previous dose by between two weeks and six weeks. For any given course of treatment the time period between each dose may or may not be a consistent period. As an illustrative example, the time period between the first and second dose may or may not be the same as the time period between the second and third dose.

In an embodiment the course of treatment comprises multiple doses administered over a total treatment period of between three and twelve months. In an embodiment the course of treatment comprises multiple doses administered over a total treatment period of between six and twelve months. In an embodiment the course of treatment comprises multiple doses administered over a total treatment period of between three and nine months. In an embodiment the course of treatment comprises multiple doses administered over a total treatment period of between six and nine months.

### Kits

The invention described herein also provides kits of components that may be for use in the methods of the invention. The invention thus provides a kit comprising, in separate containers, (i) a pharmaceutical composition comprising stern or progenitor cells, e.g. mesenchymal stern cells (MSCs), derived from a female donor animal, and (ii) a pharmaceutical composition comprising stern or progenitor cells, e.g. mesenchymal stem cells (MSCs), derived from a male donor animal. By having kits of such components available, the treating physician is able to treat either male or female patient that may present. Typically the components of the kit are stored frozen until required for administration.

The invention also provides a kit comprising, in separate containers, (i) a pharmaceutical composition comprising cell secretions from cell culture of cells derived from one or more female donor animals, and (ii) a pharmaceutical composition comprising cell secretions from cell culture of cells derived from one or more male donor animals. In an embodiment the cell secretions are derived from cell culture of stem or progenitor cells described herein. In an embodiment the cell secretions are derived from cell culture of MSCs described herein. In an embodiment the cell secretions are derived from adipose tissue-derived cells, such as cells of the stromal vascular fraction or adipocytes.

In an embodiment the kit may comprise any of the compositions described herein.

As used herein, the term “kit” refers to any delivery system for delivering materials. In the context of the detection assays and methods described herein, such delivery systems include systems that allow for the storage, transport, or delivery of reaction reagents (for example labels, reference samples, supporting material, etc. in the appropriate containers) and/or supporting materials (for example, buffers, written instructions for performing the assay, etc.) from one location to another. For example, kits include one or more enclosures, such as boxes, containing the relevant reaction reagents and/or supporting materials.

In general, the kits of the invention may comprise any number of additional components. As described herein, for example, administration of a pharmaceutical composition of cell secretions to a subject being treated for osteoarthritis and other inflammatory diseases has been demonstrated in Australian Patent Application No. 2010347212 entitled “Cell free preparation and uses thereof”, and also in WO2013/040649 entitled “Therapeutic methods and compositions”, the entire contents of both of which are incorporated herein by reference, to provide therapeutic benefit to the subject. Accordingly, a kit for use in the methods of the invention may further comprise a pharmaceutical composition comprising cell secretions. The cell secretions may be present in one or both of the pharmaceutical compositions comprising MSCs or the cell secretions may be present as a composition in a separate container or containers in the kit. The cell secretions contained in the kit may be selected as being derived from cell culture of cells of a single known sex, in which case the kit makes available to the practitioner the opportunity to administer also sex-matched cell secretions to a subject, or the cell secretions may be derived from cell culture of a mixed population of cells, or of an indeterminate population of cells.

The cell secretions may be prepared by any appropriate methods. For example, Australian Patent Application No. 2010347212 entitled “Cell free preparation and uses thereof” describes methods for generating a cell free composition of cell secretions, which method comprises culturing a population of cells comprising tissue stem cells so that there is cell replication in the population of cells comprising tissue stem cells, exposing the population of cells comprising tissue stern cells to an aqueous medium in vitro and then isolating the aqueous medium from the population of cells to produce a cell free composition. In the methods and kits of the instant invention, the cell secretions may or may not be separated from the cultured MSCs. WO2013/040649 entitled “Therapeutic methods and compositions” also describes methods by which cell secretions may be prepared for pharmaceutical use.

The invention will now be described in more detail, by way of illustration only, with respect to the following examples. The examples are intended to serve to illustrate this invention and should not be construed as limiting the generality of the disclosure of the description throughout this specification.

### EXAMPLES

### Example 1

### Preparation of Canine Adipose Derived Cells for Allogeneic Treatment

### Processing of Adipose Tissue

A 10 g sample of falciform or inguinal adipose tissue was collected from either male or female dogs. The adipose tissue was rinsed with saline and then minced finely using scissors and mixed with 20 mls of Dulbecco's Modified Eagle's Medium (DMEM, Sigma). Collagenase (Sigma) was added to a final concentration of 0.05% and the sample was incubated at 37° C. for 30 minutes. During the incubation the sample was gently mixed on an orbital shaker.

Following collagenase treatment the sample was aseptically filtered through a stainless steel mesh (700 μm pore size), transferred to a 50 ml centrifuge tube and centrifuged at 500 g for 15 minutes. The floating cells and the supernatant were discarded and the pelleted cells were gently mixed with a pasteur pipette and transferred to a 15 ml centrifuge tube.

The cells were then washed in DMEM to remove collagenase. DMEM was added to a final volume of 14 mls and the sample centrifuged at 500 g for 10 minutes. The supernatant was discarded and the pelleted SVF cells were gently resuspended in 4 mls of DMEM and mixed with a pasteur pipette.

### Expansion of Cells

Aliquots (0.5 mls) of the cell suspension were transferred to tissue culture flasks containing DMEM plus 10% canine serum and incubated in a CO2 incubator at 37° C. until a confluent cell monolayer was present (7 to 10 days). Cells were stripped with 3 mls of TrypLE Express (Invitrogen), decanted into 50 ml centrifuge tubes and centrifuged at 500× g for 10 minutes. Cells were passaged further until they had doubled approximately 8 or 13 times. The passaged cells were then stripped and centrifuged.

### Cryopreservation of Cells

The pelleted cell samples were mixed with CryoStor (Stemcell Technologies, Tullamarine, Australia), aliquoted into cryogenic vials and cryopreserved in a Mr Frosty slow freezing device (Invitrogen) in a −80° C. freezer for 24 hours and then transferred to a liquid nitrogen dewar.

### Example 2

### Production of Secretions from Passaged Cells

Canine adipose derived cells were isolated and cultured as described in Example 1. The cells were passaged until the cells had reached a cumulative cell doubling of approximately 13 times. The tissue culture supernatant from the cells was concentrated in a 3 kDa Amicon (Millipore) and stored frozen.

### Example 3

### Preparation of a Mixture of Canine Adipose Derived Cells and Cell Secretions for Allogeneic Treatment

Canine adipose derived cells were isolated and cultured as described in Example 1. Prior to freezing the cells were mixed with concentrated canine secretions produced as described in Example 2 mixed 1:1 with canine serum. The cells were stripped, washed and the cell pellet was resuspended in the mixture of serum and secretions and then held at room temperature for 30 minutes to allow the secretions to interact with the cells. The cell suspensions were then transferred to cryovials, mixed with DMSO and frozen as described in Example 1.

### Example 4

### Treatment of Dogs for Elbow Osteoarthritis in a Blinded Placebo Controlled Trial with Female Canine Adipose Derived Cells

### Preparation of Cells

Cells were produced from a female dog as described in Example 1.

### Treatment of Dogs

Five male dogs and 5 female dogs with osteoarthritis of the elbow and stifle were treated with a single intra-articular injection of 2.7 million cells. A placebo group of 10 dogs (6 males and 3 females) received a sham injection. The dog owners and consulting vets were not informed if their dog was in the treatment or placebo group.

Assessment of dogs

The Canine Brief Pain Inventory (CBPI) was used to assess the response to treatment. CBPI is an owner questionnaire that generates Pain Severity Scores (PSS) and Pain Inference Scores (PIS). The PSS is a set of 4 questions which asks the owner to score the severity of the dog's pain, at its worst, at its best, as it is currently and on average in the last 7 days. The PIS is a set of 6 questions that asks the owner to score the level of pain which interferes with dogs routine functioning. Examples include the ability to get up from lying down, the ability to run, walk, jump and climb stairs. The scoring system has been fully validated and is accepted by the US FDA as an appropriate means for assessment of new drugs.

Dogs were assessed two weeks prior to treatment, on the treatment day and 10 days, 1, 2 3 month and 6 months post treatment. Scores to 2 months were available for analysis. The scores from two weeks prior to treatment and on the treatment day were averaged to generate a baseline score.

### Results

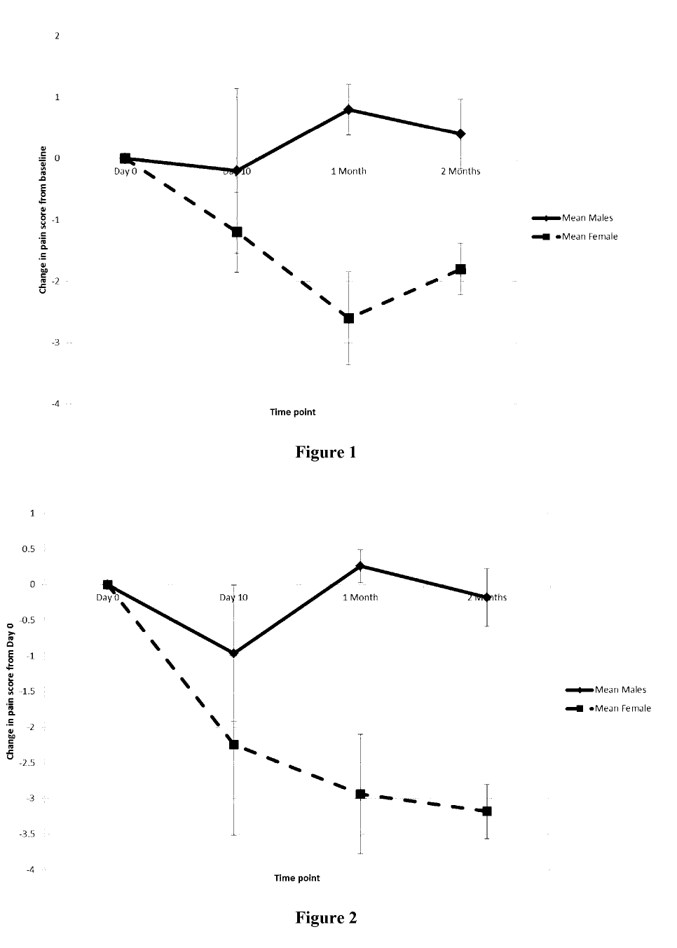

The results from treating the 10 dogs with female cells are presented in FIGS. 1 and 2. There was a larger therapeutic effect in female dogs treated with female cells than with male dogs treated with female cells.

The average PSS for male dogs showed a marginal improvement at day 10 and was worse than pretreatment at 1 month and 2 months. The average PSS for the female dogs showed a moderate improvement at day 10 and a large improvement at 1 month and 2 months.

The average PIS for male dogs showed an improvement at day 10 and was worse than pretreatment at 1 month and marginally improved at 2 months. The average PIS for female dogs showed a large improvement at day 10 and a very large improvement at 1 month and 2 months.

### Example 5

### Treatment of Dogs for Osteoarthritis in an Open Trial with Female Canine Adipose Derived Cells

### Administration of Cells

Fifty-three dogs with osteoarthritis were treated with female cells mixed with cell secretions from female cells prepared as described in Example 3. There was no control group.

### Assessment of Dogs

The Canine Brief Pain Inventory (CBPI) was used to assess the response to treatment. CBPI is an owner questionnaire that generates Pain Severity Scores (PSS) and Pain Inference Scores (PIS).

Dogs were assessed on the treatment day and 10 days, 1 month, 2 months and 3 months post treatment. Not every dog was assessed at all time points.

### Results

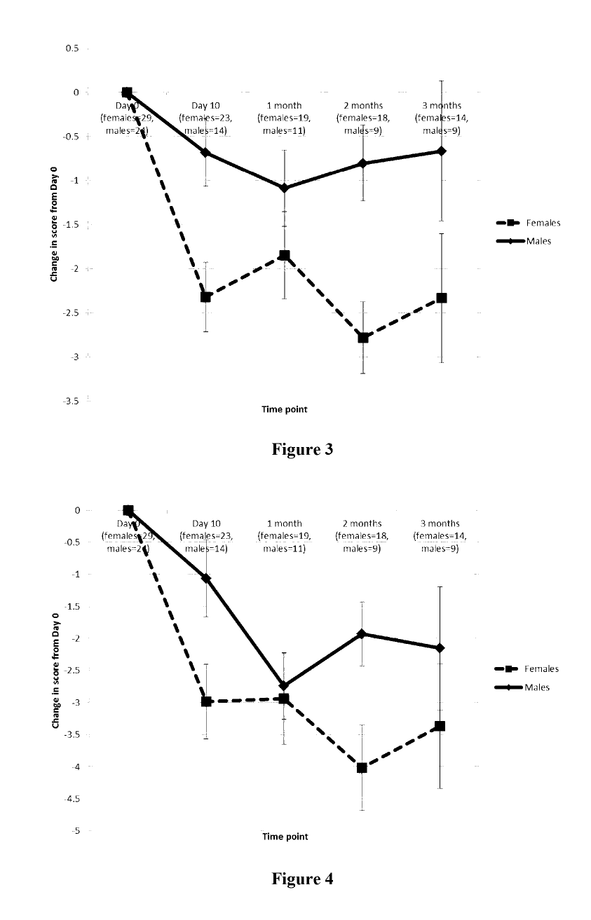

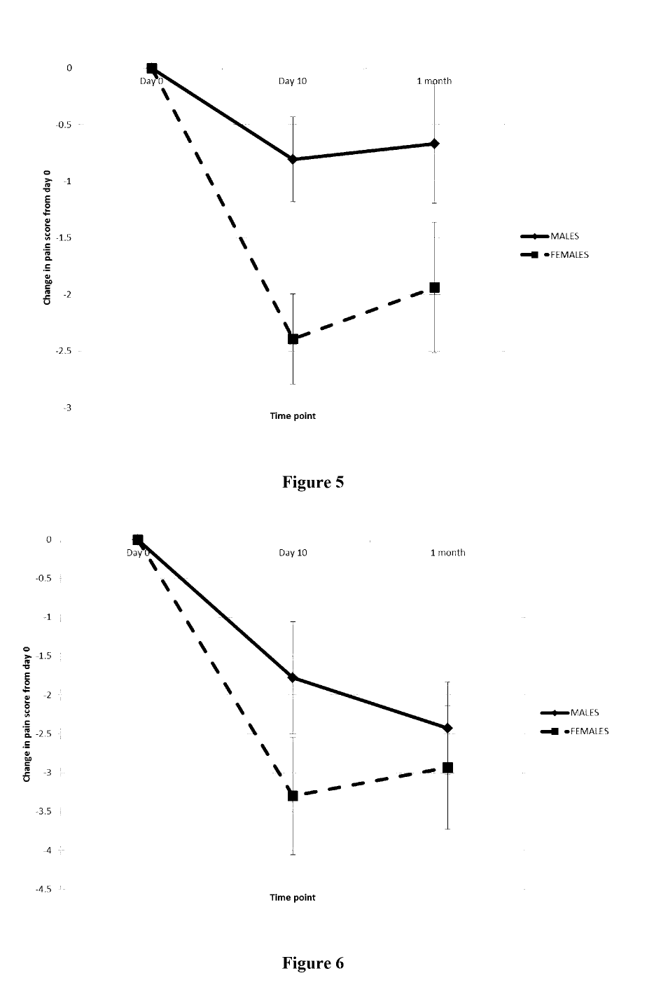

The average PSS and PIS for all dogs treated in the open trial are presented in FIGS. 3 and 4. Note that not all owners completed the questionnaires at all time points. Owner questionnaires were completed for 25 of the 53 dogs for the first 3 time points. The average PSS and PIS for these 25 dogs are presented in FIGS. 5 and 6.

FIGS. 3, 4, 5 and 6 show that female dogs showed a larger response to treatment than male dogs.

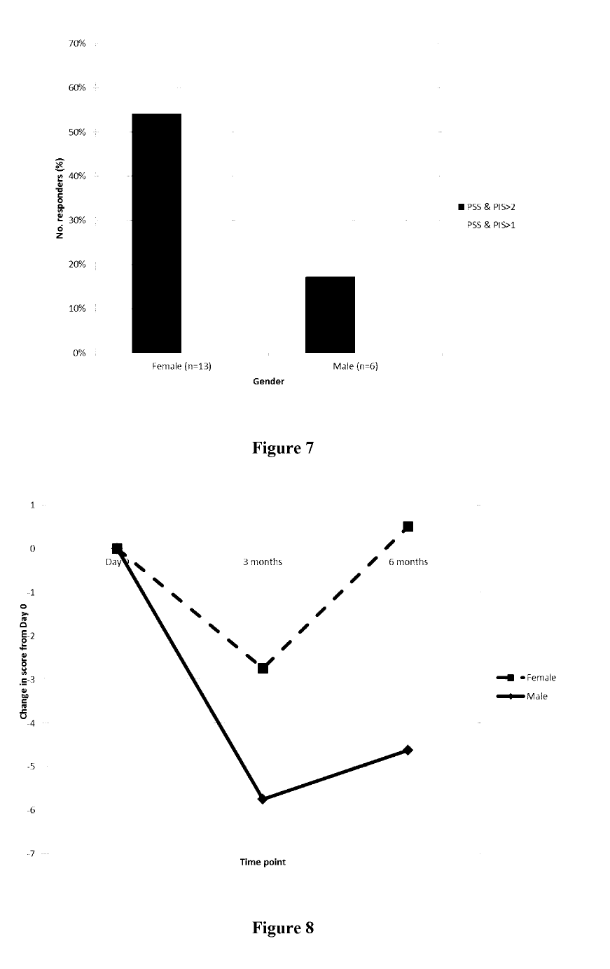

FIG. 7 shows the percentage of the 53 dogs treated that responded to treatment with an improvement of greater than 1 and greater than 2 in PSS and PIS. Considerably more female dogs responded than male dogs.

### Example 6

### Treatment of Dogs for Osteoarthritis in an Open Trial with Male Canine Adipose Derived Cells

Administration of cells

Four dogs with osteoarthritis were treated with male cells prepared as described in Example 1. There was no control group.

Assessment of dogs

The Canine Brief Pain Inventory (CBPI) was used to assess the response to treatment. CBPI is an owner questionnaire that generates Pain Severity Scores (PSS) and Pain Inference Scores (PIS).

Dogs were assessed on the treatment day and 10 days, 3 months and 6 months post treatment. Not every dog was assessed at all time points.

### Results

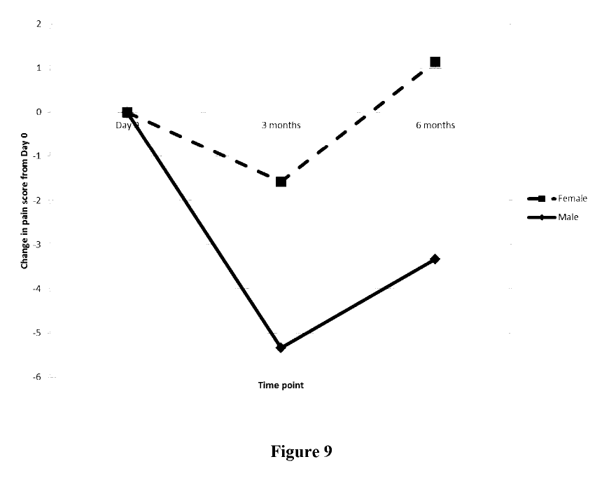

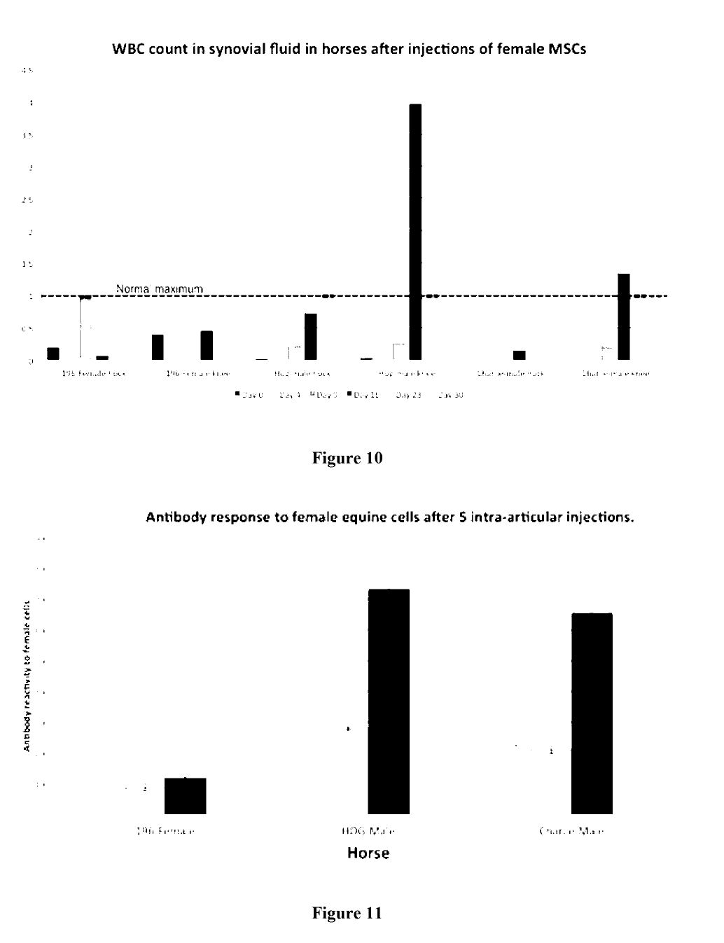

The average PSS and PIS for dogs treated with male cells are presented in FIGS. 8 and 9. Note that not all owners completed the questionnaires at all time points. Owner questionnaires were completed for 1 or 2 dogs at each time point. The average PSS and PIS for these 1-2 dogs are presented in FIGS. 8 and 9. The male dogs showed a larger response to treatment than female dogs.

The Examples herein suggest that the use of sex-matched donor cells for the treatment of an inflammatory disorder, exemplified by osteoarthritis, is advantageous for the treatment of pain.

### Example 7

### Immune Response to Sex Mismatched Cells in Horses

### Production of Allogeneic MSCs

A 10 g sample of adipose tissue was collected from the tail base of a female horse. The adipose tissue was rinsed with saline and then minced finely using scissors and mixed with 20 mls of Dulbecco's Modified Eagle's Medium (DMEM, Sigma). Collagenase (Sigma) was added to a final concentration of 0.05% and the sample was incubated at 37° C. for 90 minutes. During the incubation the sample was gently inverted by hand every 15 minutes.

Following collagenase treatment the sample was aseptically filtered through a stainless steel mesh (700 gm pore size), transferred to a 50 ml centrifuge tube and centrifuged at 500 g for 15 minutes. The supernatant was discarded and the pelleted cells resuspended in DMEM. The cells were centrifuged again and resuspended in DMEM.

The cell suspension were transferred to a T175 tissue culture flask containing 50 mls of DMEM plus 10% canine serum and incubated in a CO2 incubator at 37° C. until a confluent cell monolayer was present (6 days). Cells were stripped with 3 mls of TrypLE Express (Invitrogen) and transferred to two T175 tissue culture flasks. Cells were passaged a further 3 times and then stripped, harvested and resuspended in a cryopreservation solution and aliquoted in to cryovials with 3 millions cells per vial. The cells were frozen in a controlled rate freezer and then stored in a liquid nitrogen dewar.

### Horses

Two male horses, a stallion and a gelding, and a healthy female horse were injected with MSCs in to the hock and knee joints. All three horses were healthy, in that none was suffering from any obvious disorder or disease. A vial of MSCs was thawed and the entire contents injected in to the knee joint. A second vial was injected to the hock joint. Injections were repeated five times at weekly intervals.

Blood and synovial fluid was collected from the horses immediately before each injection. Synovial fluid was sent to a veterinary pathology laboratory for enumeration of white blood cells (WBCs). Blood was collected in to clotting tubes and the serum stored frozen until analysis.

### Clinical Signs

One of the male horses, the gelding, developed a flare in the hock joint after the first injection. The flare resolved after 1 week. There were no further clinical signs of response to the injections.

### Analysis of Synovial Fluid

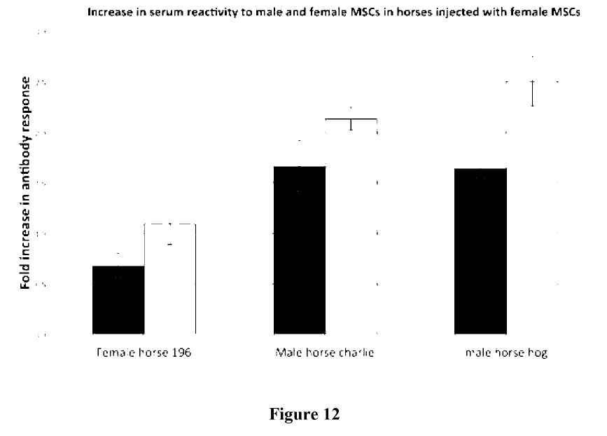

The WBC counts from the synovial fluid, after each injection are shown in FIG. 10. The gelding (male) had WBC counts well above the normal maximum range in both joints. The stallion (male) had WBC counts well above the normal maximum range three times in the knee joint. The female horse did not have a count above the normal maximum range in either joint at any time point. Counts above the normal maximum range indicate an inflammatory event within the joint.

### Analysis of Serum

The horse serum was analysed for antibodies against the female MSCs. The serum was diluted in PBS plus 1% canine serum and mixed with female MSCs from the same batch as those used for the injections. After incubation at room temperature for 30 minutes the cells were washed by centrifuging at 5000 g for 5 minutes and the supernatant discarded. Cells were resuspended in PBS plus 1% canine serum and centrifuged again. The supernatant was discarded and the cells resuspended in PBS plus 1% canine serum. A anti-horse FITC labeled antibody (Sigma. Chemical Company) was added to the cells and allowed to incubate for 60 minutes at room temperature. The samples were then analysed by flow cytometry and the fluorescence intensity of the cells recorded. The staining and analysis was repeated three times.

The results are presented in FIG. 11. The histograms represent the average fluorescent intensity of cells from 3 replicate analysis of 2000 cells. The error bars represent the standard deviation.

The male horses showed a large increase in the fluorescence of the stained cells between pretreatment and post treatment serum samples. This reflects an increase in antibodies that are reactive to the cells. The female horse did not show a similar increase in antibodies to the cells.

The serum was further analysed for antibodies to both female and male MSCs. The serum was analysed as described above but reacted with both male and female adipose derived horse MSCs. Cells were analysed by flow cytomtery and the pre and post injection samples compared. Results are displayed in FIG. 12 as fold increase in antibody response after the injections. A result of >1 indicates an increase in antibody response after five injections of female cells.

The two male horses showed a >2 fold increase in antibody response to female cells and a lesser increase in antibody response to male cells. The female horses showed no increase in antibody response to either female or male cells. This would suggest that the male horses are making some antibodies that are specific to the female cells.

### Example 8

### Immune Response to Male Cells in a Female Horse

One female horse was injected in to one hock and one knee joint with male adipose derived cells prepared as described in Example 7. Injections were repeated weekly for 5 weeks. The horse was monitored for joint flaring, effusion and lameness.

After the second injection the hock and the knee joint flared with signs of effusion and lameness.

### Example 9

### No Immune Response to Female Cells in Female Horses

Nine female horses were injected in to one hock and one knee joint with female cells prepared as described in Example 7. Injections were repeated weekly for 5 weeks. The horses were monitored for joint flaring, effusion and lameness. None of the nine horses developed a joint flare or showed any joint effusion or lameness.

These results are clearly different to the response observed when a female horse was injected with male cells as described in Example 8. It is clear that injection of sex-mismatched cells causes an inflammatory response, whereas injections of sex matched cells does not cause an inflammatory response.

### Example 10

### Treatment of Heart Disease with Sex-Matched Cells

Preparation of cells

Bone marrow (20 ml) was collected from the sternum of a male and female horse and mixed with 10 mM EDTA. The nucleated cells were purified by density gradient centrifugation with Ficol. The nucleated cells were cultured as in Example 1 except that the serum used in the culture media was 10% fetal calf serum. The cells became confluent after 7 days and were passaged three times. Cells were harvested and stored frozen as described in Example 1.

### Treatment of Horses

Two male and two female horses with ventricular premature complexes (VPC) and one female horse with low grade atrial fibrillation brought on by exertion, were given an IV injection of sex-matched cells.

### Results of Treatment

VPC cases on a scale of 0 to 5. Case 1—female: improved one grade from 2/5 to 1/5. Case 2—female: no response, remained 1/5. Case 3—male: improved one grade from 3/5 to 2/5. Case 4—male: improved two grades from 3/5 to 1/5. The female horse with low-grade atrial fibrillation did not show any change.

1. A method for treatment of an inflammatory disorder in a subject, the method comprising the steps of: identifying the sex of the subject; selecting a pharmaceutical composition comprising stem or progenitor cells derived from one or more individuals of the same sex as the subject; and administering to said subject a therapeutically effective amount of the selected sex-matched cell composition.

2. A method for treatment of an inflammatory disorder in a subject, the method comprising the steps of: identifying the sex of the subject; selecting a pharmaceutical composition comprising cell secretions from cell culture of cells derived from one or more individuals of the same sex as the subject; and administering to said subject a therapeutically effective amount of the selected sex-matched cell secretions composition.

3. The method according to claim 1 or 2, wherein the cells are mesenchymal stem cells (MSCs)

4. The method according to any one of claims 1 to 3, wherein the inflammatory disorder is a joint-related inflammatory disorder, such as arthritis or osteoarthritis.

5. The method according to any one of claims 1 to 4, wherein the subject is a female.

6. The method according to any one of claims 1 to 4, wherein the subject is a male.

7. The method according to claim 3, wherein the MSCs are allogeneic to the subject.

8. The method according to claim 1, wherein the pharmaceutical composition comprises a homogeneous composition of MSCs.

9. The method according to claim 3, wherein the MSCs are culture expanded cells.

10. The method according to claim 3, wherein the MSCs are derived from adipose tissue, bone marrow, placenta, blood, or cord blood.

11. The method according to claim 3, wherein the MSCs are derived from multiple donor animals of the same species and sex.

12. The method according to claim 3, wherein the MSCs are derived from a de-sexed donor animal.

13. The method according to claim 12, wherein the de-sexed donor animal is a female dog.

14. The method according to any one of claims 1 to 12, wherein the subject is a human subject.

15. The method according to any one of claims 1 to 12, wherein the subject is a non-human animal, preferably selected from the group consisting of ovine, bovine, equine, porcine, feline, canine, primate, and rodent.

16. The method according to claim 15, wherein the non-human subject is a dog.

17. The method according to any one of claims 1 to 12, wherein the subject is a de-sexed non-human female animal.

18. The method according to any one of claims 1 to 12, wherein the subject is a de-sexed non-human male animal.

19. The method according to claim 1, wherein the subject is administered a therapeutically effective amount of a pharmaceutical composition comprising cell secretions.

20. The method according to claim 19, wherein the cell secretions are derived from cell culture of cells derived from one or more individuals of the same sex as the recipient subject.

21. The method according to claim 19 or 20, wherein the cells and the cell secretions are together in one pharmaceutical composition.

22. The method according to claim 2, wherein the subject is administered a therapeutically effective amount of a pharmaceutical composition comprising sex-matched stem or progenitor cells.

23. The method according to any one of claims 19 to 22, wherein the cells are mesenchymal stem cells (MSCs).

24. A kit comprising, in separate containers, (i) a pharmaceutical composition comprising stem or progenitor cells derived from a female donor animal, and (ii) a pharmaceutical composition comprising stem or progenitor cells derived from a male donor animal.

25. A kit comprising, in separate containers, (i) a pharmaceutical composition comprising cell secretions from cell culture of cells derived from one or more female donor animals, and (ii) a pharmaceutical composition comprising cell secretions from cell culture of cells derived from one or more male donor animals.

26. The kit according to claim 24, wherein the kit further comprises instructions for sex-matched administration of a pharmaceutical composition comprising stem or progenitor cells to a subject having an inflammatory disorder.

27. The kit according to claim 25, wherein the kit further comprises instructions for sex-matched administration of a pharmaceutical composition comprising cell secretions to a subject having an inflammatory disorder.

28. The kit according to any one of claims 24 to 27, wherein the inflammatory disorder is a joint-related inflammatory disorder, such as arthritis or osteoarthritis.

29. The kit according to any one of claims 24 to 28, wherein the cells are mesenchymal stem cells (MSCs).

30. The kit according to claim 24, wherein the pharmaceutical composition is a cryopreserved pharmaceutical composition of MSCs.

31. The kit according to claim 24, wherein the pharmaceutical composition comprises a homogeneous composition of MSCs.

32. The kit according to any one of claims 29 to 31, wherein the MSCs are culture expanded cells.

33. The kit according to any one of claims 29 to 32, wherein the MSCs are derived from adipose tissue, bone marrow, placenta, blood, cord tissue or cord blood.

34. The kit according to any one of claims 29 to 33, wherein the MSCs derived from a female donor animal and the MSCs derived from a male donor animal are animals of the same species.

35. The kit according to any one of claims 29 to 34, wherein the MSCs in a composition are derived from multiple donor animals of the same species and sex.

36. The kit according to any one of claims 29 to 35, wherein the MSCs are derived from a de-sexed donor animal.

37. The kit according to any one of claims 29 to 36, wherein the de-sexed donor animal is a female dog.

38. The kit according to any one of claims 24 to 36, wherein the donor animal is a human.

39. The kit according to any one of claims 24 to 36, wherein the donor animal is a non-human animal.

40. The kit according to claim 39, wherein the non-human donor animal is selected from the group consisting of ovine, bovine, equine, porcine, feline, canine, primate, and rodent.

41. The kit according to claim 40, wherein the non-human donor animal is a dog.

42. The kit according to claim 24, wherein one or more of the pharmaceutical composition(s) comprising stem or progenitor cells further comprises cell secretions.

43. The kit according to claim 24, wherein the kit further comprises a pharmaceutical composition comprising cell secretions.

44. The kit according to claim 42 or 43, wherein the cell secretions are derived from cell culture of cells derived from one or more individuals of the same sex as the recipient subject.

45. The kit according to claim 42, wherein the cell secretions and the stem or progenitor cells are together in one pharmaceutical composition.

46. The kit according to claim 45, wherein the cell secretions and the stem or progenitor cells together in a pharmaceutical composition are derived from one or more donors of the same sex.

47. The kit according to any one of claims 42 to 46 wherein the stem cells are MSCs.

48. A method for the treatment of pain associated with osteoarthritis in a subject, the method comprising the steps of: identifying the sex of the subject; selecting a pharmaceutical composition comprising mesenchymal stem cells (MSCs) derived from one or more individuals of the same sex as the subject; and administering to said subject a therapeutically effective amount of the selected sex-matched cell composition.

49. A method for reducing an adverse immune response in a subject administered sex mismatched MSCs, the method comprising (i) administering to said subject (i) one or more immune suppressant drug(s) and or (ii) conditioned media from cell culture of stem cells.

50. The method of claim 39, wherein said subject is male.

51. A method for the treatment of pain associated with an inflammatory disorder in a subject, the method comprising the steps of: identifying the sex of the subject; selecting a pharmaceutical composition comprising stem or progenitor cells derived from one or more individuals of the same sex as the subject; and administering to said subject a therapeutically effective amount of the selected sex-matched cell composition.

52. A method for treatment of pain associated with an inflammatory disorder in a subject, the method comprising the steps of: identifying the sex of the subject; selecting a pharmaceutical composition comprising cell secretions from cell culture of cells derived from one or more individuals of the same sex as the subject; and administering to said subject a therapeutically effective amount of the selected sex-matched cell secretions composition.

53. A method for reducing an adverse immune response in a subject administered sex mismatched stem or progenitor cells or cell secretions from culture of such cells, the method comprising (i) administering to said subject (i) one or more immune suppressant drug(s) and or (ii) conditioned media from cell culture of stem cells. In an embodiment the subject is a male animal.

54. The method according to any one of claims 1 to 23 and 48 to 53, wherein the method is a non-autologous method.
